(19) United States
(12) Patent Application Publication
(10) Pub. No.: US 2018/0177411 A1
(43) Pub. Date: Jun. 28, 2018

(54) BLOOD PRESSURE MEASUREMENT INSTRUMENT, AND KOROTKOFF'S SOUND RECOGNITION METHOD AND DEVICE

(71) Applicant: BOE TECHNOLOGY GROUP CO., LTD., Beijing (CN)

(72) Inventors: Hui DU, Beijing (CN); Zuo YUAN, Beijing (CN)

(21) Appl. No.: 15/737,055

(22) PCT Filed: May 10, 2017
(86) PCT No.: PCT/CN2017/083712

§ 371 Date: Dec. 15, 2017

(30) Foreign Application Priority Data
May 12, 2016 (CN) 201610317930.9

(51) Int. Cl. A61B 5/0225; A61B 5/022; A61B 5/00; A61B 5/0235

(52) U.S. Cl. CPC A61B 5/0225; A61B 5/02208; A61B 5/0235; A61B 5/02233; A61B 5/7275

(57) ABSTRACT

A blood pressure measurement instrument includes: a cuff capable of being worn at a measured position on a body of a user, a pressure sensor, a sound sensor, a pressure regulator, and a processor. The pressure regulator is configured to regulate a pressure applied to the measured position by the cuff; the pressure sensor is configured to acquire the pressure applied to the measured position by the cuff during a period of time when the pressure is changed; the sound sensor is positioned between the cuff and the measured position in a measuring process and is configured to acquire blood flow sound at the measured position; and the processor is connected with the pressure sensor and the sound sensor and is configured to determine a blood pressure value of the user according to acquisition results of the pressure sensor and the sound sensor.

### TECHNICAL FIELD

Embodiments of the present disclosure relate to the technical field of medical apparatus and instruments, and particularly relate to a blood pressure measurement instrument and a Korotkoff sound identifying method and apparatus.

### BACKGROUND

In recent years, along with constant deepening of cognition of people on effects of multiple risk factors of cardiovascular diseases and protection of organs of heart, brain, kidney and the like, people pay more and more attention to prevention, diagnosis and treatment of hypertension. Hypertension patients need to pay close attention to monitoring and management of blood pressures at ordinary times, and control blood pressures in a mode of medicine treatment and the like according to fluctuation of the blood pressures.

Currently common non-invasive blood pressure measurement methods are mainly divided into a Korotkoff sound method (a mercury auscultation method) and an oscillatory method. The oscillatory method is that according to a hemodynamic principle of heartbeat, in the process that after the brachial artery vessel blocks up blood flow via external force compression, decompression is gradually carried out to enable the blood flow to break through the vessel again to flow, pressure fluctuation which is synchronous with the heartbeat, i.e., a pulse wave, is acquired and recorded, and according to a relationship between an amplitude of the pulse wave and a pressure at the position of the brachial artery vessel, the blood pressure is estimated.

Currently, electronic sphygmomanometers in the market are all designed on the basis of the principle of the oscillatory method. The electronic sphygmomanometer has the advantages of high measurement stability, simplicity for operation, convenience for use and the like, but judgment of the oscillatory method on a blood pressure value is mainly based on experience and a statistical law, there is no standardization algorithm and in the using process, the oscillatory method may generate a large deviation at times, and thus, measurement accuracy of the oscillatory method is not generally approved in medicine.

The Korotkoff sound method is found for the first time in 1905 by Doctor Korotkoff of the Soviet Union. After the brachial artery vessel blocks up blood flow via external force compression, decompression is gradually carried out, and along with reduction of the pressure, a set of friction and impact sound, i.e., Korotkoff sound, which is produced after the blood flow breaks through the vessel again, gradually becomes strong from appearance, then is gradually weakened to disappear and is synchronous with pulse, can be heard from a stethoscope arranged at the position of the brachial artery. After the blood flow breaks through the vessel again, when the Korotkoff sound appears, a corresponding mercury pressure value is a systolic blood pressure, and when the Korotkoff sound is gradually weakened to disappear, a corresponding mercury pressure value is a diastolic blood pressure. The Korotkoff sound method is the blood pressure measurement gold standard which is currently recognized in medicine, but a mercury stethoscope which applies the Korotkoff sound method to measure the blood pressure requests an operator to observe change of a mercury pressure gauge while listening to sound by the stethoscope and read a mercury pressure value while distinguishing the Korotkoff sound, subjective judgment of the operator much influences a measurement result, and different operators obtain different results in the measurement so as to cause a case that an accurate measured value possibly cannot be obtained.

### SUMMARY

At least one embodiment of the present disclosure provides a blood pressure measurement instrument, comprising: a cuff capable of being worn at a measured position on a body of a user, a pressure sensor, a sound sensor, a pressure regulator and a processor; the pressure regulator is connected with the cuff, and is configured to regulate a pressure applied to the measured position by the cuff; the pressure sensor is arranged on the cuff, and is configured to acquire the pressure applied to the measured position by the cuff during a period of time when the pressure is changed; the sound sensor is positioned between the cuff and the measured position in a measuring process, and is configured to acquire blood flow sound at the measured position; and the processor is communicated with the pressure sensor and the sound sensor, and is configured to determine a blood pressure value of the user according to acquisition results of the pressure sensor and the sound sensor.

In an embodiment of the present disclosure, the blood flow sound includes Korotkoff sound, and the blood pressure measurement instrument is configured to acquire pressure values respectively corresponding to an appearance moment and a disappearance moment of the Korotkoff sound during the period of time when the pressure is changed so as to determine the blood pressure value.

In an embodiment of the present disclosure, the processor includes a Korotkoff sound processing unit, the Korotkoff sound processing unit including a peak value acquiring module and a peak value comparing module; the peak value acquiring module is configured to acquire a sound intensity peak value of the Korotkoff sound at the measured position in each period; and the peak value comparing module is connected with the peak value acquiring module, and is configured to, according to acquisition results of the peak value acquiring module, compare a peak value in a current period with a peak value in a previous period.

In an embodiment of the present disclosure, the Korotkoff sound processing unit further includes a maximum peak value determining module, the maximum peak value determining module being connected with the peak value comparing module and configured to, when the peak value comparing module concludes that the peak value in the current period is smaller than the peak value in the previous period for the first time, determine the peak value in the previous period as a maximum peak value of the Korotkoff sound, wherein the period in which the maximum peak value of the Korotkoff sound is determined is referred to as a reference period.

In an embodiment of the present disclosure, the Korotkoff sound processing unit further includes a node determining module, the node determining module being connected with the peak value comparing module and configured to, when the peak value comparing module concludes that the peak value in the current period is greater than or equal to 105% to 110% of the peak value in the previous period for the first time, determine a moment when the peak value in the current period appears as an appearance moment of the Korotkoff sound.

In an embodiment of the present disclosure, the node determining module is further connected with the maximum peak value determining module, and is configured to, when the peak value comparing module compares a peak value in each period after the reference period with the maximum peak value of the Korotkoff sound until the peak value comparing module obtains one period in which a peak value of this one period is smaller than or equal to 65% to 75% of the maximum peak value of the Korotkoff sound, determine a moment when the peak value in the one period appears as a disappearance moment of the Korotkoff sound.

In an embodiment of the present disclosure, the processor further includes a pressure matching unit; and the pressure matching unit is connected with the Korotkoff sound processing unit and the pressure sensor, and is configured to receive the appearance moment and the disappearance moment of the Korotkoff sound, which are output by the Korotkoff sound processing unit, acquire a pressure value acquired by the pressure sensor at the appearance moment of the Korotkoff sound as a systolic blood pressure and acquire a pressure value acquired by the pressure sensor at the disappearance moment of the Korotkoff sound as a diastolic blood pressure.

In an embodiment of the present disclosure, the pressure regulator includes an air pump, an air delivery pipe connected with the air pump and an airbag connected to the other end of the air delivery pipe, the airbag is arranged inside the cuff, and the air pump is configured to inflate and deflate the airbag by the air delivery pipe.

In an embodiment of the present disclosure, the processor further includes a pressure controlling unit; and the pressure regulator further includes a regulation valve mounted on the air pump, and the regulation valve is connected with the pressure controlling unit and is configured to regulate a pressure in the air pump under control of the pressure controlling unit.

In an embodiment of the present disclosure, the regulation valve is configured to regulate the air pump to pressurize the airbag at a speed of 2 to 4 mmHg/s under control of the pressure controlling unit; or, when an air pressure in the airbag reaches 210 mmHg to 260 mmHg, regulate the air pump to release the pressure in the airbag at a speed of 2 to 4 mmHg/s; or, in two to three seconds after the processor determines the disappearance moment of the Korotkoff sound, regulate the air pump to release the pressure in the airbag at a speed of 8 to 12 mmHg/s.

In an embodiment of the present disclosure, the cuff further includes connecting pieces arranged at both ends of the cuff, and when the cuff is wound at the measured position, the connecting pieces are used for fixing both ends of the cuff.

In an embodiment of the present disclosure, the sound sensor is mounted on one side of the cuff, which side is close to the measured position.

At least one embodiment of the present disclosure provides a Korotkoff sound identifying method for acquiring blood flow sound including Korotkoff sound at a measured position, comprising: acquiring a sound intensity peak value of the Korotkoff sound in each period; comparing a peak value in a current period with a peak value in a previous period; regulating a pressure at the measured position; and determining an appearance moment, a maximum peak value and a disappearance moment of the Korotkoff sound.

In an embodiment of the present disclosure, when the peak value in the current period is greater than or equal to 105% to 110% of the peak value in the previous period for a first time, a moment when the peak value in the current period appears is determined as the appearance moment of the Korotkoff sound.

In an embodiment of the present disclosure, when the peak value in the current period is smaller than the peak value in the previous period for the first time, the peak value in the previous period is determined as the maximum peak value of the Korotkoff sound, wherein a period in which the maximum peak value of the Korotkoff sound is determined is referred to as a reference period.

In an embodiment of the present disclosure, in a case of comparing a peak value in each period after the reference period with the maximum peak value of the Korotkoff sound, when one period in which a peak value of this one period is smaller than or equal to 65% to 75% of the maximum peak value of the Korotkoff sound is compared out, a moment when the peak value in the one period appears is determined as a disappearance moment of the Korotkoff sound.

In an embodiment of the present disclosure, the process that when the peak value in the current period is smaller than the peak value in the previous period for the first time, the peak value in the previous period is determined as the maximum peak value of the Korotkoff sound, wherein the period in which the maximum peak value of the Korotkoff sound is determined is referred to as the reference period includes: when the peak value in the current period is smaller than the peak value in the previous period for the first time, referring to the previous period as a first period, and referring to the current period as a second period; after the second period, at least carrying out an operation of comparing a peak value in a new current period with a peak value in a new previous period; and when a comparison result of the above operation each shows that the peak value in the new current period is smaller than the peak value in the new previous period, determining the first period as the reference period.

In an embodiment of the present disclosure, before the appearance moment of the Korotkoff sound, regulating the pressure at the measured position includes: pressurizing the measured position at a speed of 2 to 4 mmHg/s; and when the pressure at the measured position reaches 210 mmHg to 260 mmHg, releasing the pressure at the measured position at a speed of 2 to 4 mmHg/s; or, in two to three seconds after the disappearance moment of the Korotkoff sound, regulating the pressure at the measured position further includes: releasing the pressure at the measured position at a speed of 8 to 12 mmHg/s.

At least one embodiment of the present disclosure provides an apparatus for carrying out Korotkoff sound identification by adopting the Korotkoff sound identifying method, comprising a Korotkoff sound processing unit, the Korotkoff sound processing unit including a peak value acquiring module, a peak value comparing module, a maximum peak value determining module and a node determining module; the peak value acquiring module is configured to acquire a sound intensity peak value of Korotkoff sound at a measured position in each period; the peak value comparing module is connected with the peak value acquiring module, and is configured to, according to acquisition results of the peak value acquiring module, compare a peak value in a current period with a peak value in a previous period; the maximum peak value determining module is connected with the peak value comparing module, and is configured to determine a maximum peak value of the Korotkoff sound; the node determining module is connected with the peak value comparing module, and is configured to determine an appearance moment of the Korotkoff sound; and the node determining module is further connected with the maximum peak value determining module, and is configured to determine a disappearance moment of the Korotkoff sound.

In an embodiment of the present disclosure, the maximum peak value determining module is configured to, when the peak value comparing module concludes that the peak value in the current period is smaller than the peak value in the previous period for the first time, determine the peak value in the previous period as a maximum peak value of the Korotkoff sound, wherein the period in which the maximum peak value of the Korotkoff sound is determined is referred to as a reference period.

In an embodiment of the present disclosure, the node determining module is configured to, when the peak value comparing module concludes that the peak value in the current period is greater than or equal to 105% to 110% of the peak value in the previous period for the first time, determine a moment when the peak value in the current period appears as the appearance moment of the Korotkoff sound.

In an embodiment of the present disclosure, the node determining module is configured to, when the peak value comparing module compares a peak value in each period after the reference period with the maximum peak value of the Korotkoff sound until the peak value comparing module obtains one period in which a peak value of this one period is smaller than or equal to 65% to 75% of the maximum peak value of the Korotkoff sound, determine a moment when the peak value in the one period appears as the disappearance moment of the Korotkoff sound.

In an embodiment of the present disclosure, the apparatus further comprises a pressure controlling unit, the pressure controlling unit being configured to: before the appearance moment of the Korotkoff sound, pressurize the measured position at a speed of 2 to 4 mmHg/s; when a pressure at the measured position reaches 210 mmHg to 260 mmHg, release the pressure at the measured position at a speed of 2 to 4 mmHg/s; and in two to three seconds after the disappearance moment of the Korotkoff sound, release the pressure at the measured position at a speed of 8 to 12 mmHg/s.

### BRIEF DESCRIPTION OF THE DRAWINGS

In order to clearly illustrate the technical solution of the embodiments of the disclosure or the prior art, the drawings of the embodiments or description in the prior art will be briefly described in the following. It is obvious that the described drawings are only related to some embodiments of the disclosure, and those skilled in the art also can obtain other drawings, without any inventive work, according to the drawings.

FIG. 1 is a structural schematic diagram of a blood pressure measurement instrument provided by an embodiment of the present disclosure;

FIG. 2 is a waveform diagram of sound signals of blood flow sound acquired by a sound sensor according to an embodiment of the present disclosure;

FIG. 3 is a structural schematic diagram of a processor of a blood pressure measurement instrument provided by an embodiment of the present disclosure;

FIG. 4 is a structural schematic diagram of a pressure regulator of a blood pressure measurement instrument provided by an embodiment of the present disclosure;

FIG. 5 is a structural schematic diagram of another pressure regulator of a blood pressure measurement instrument provided by an embodiment of the present disclosure;

FIG. 6 is a structural schematic diagram of a cuff of a blood pressure measurement instrument provided by an embodiment of the present disclosure;

FIG. 7 is a structural schematic diagram of a Korotkoff sound processing unit in a processor of a blood pressure measurement instrument provided by an embodiment of the present disclosure;

FIG. 8 is a schematic diagram of a working process of a blood pressure measurement instrument provided by an embodiment of the present disclosure;

FIG. 9a to FIG. 9b are flow charts of a Korotkoff sound identifying method provided by an embodiment of the present disclosure; and

FIG. 10 is a detailed flow chart of a method to which the step S104 in FIG. 9b relates.

### DETAILED DESCRIPTION

The technical solution of the embodiments of the present disclosure will be described in a clearly and fully understandable way in connection with the drawings. It is obvious that the described embodiments are just a part but not all of the embodiments of the disclosure. Based on the embodiments of the disclosure, those skilled in the art can obtain other embodiment(s), without any inventive work, which should be within the scope of protection of the disclosure.

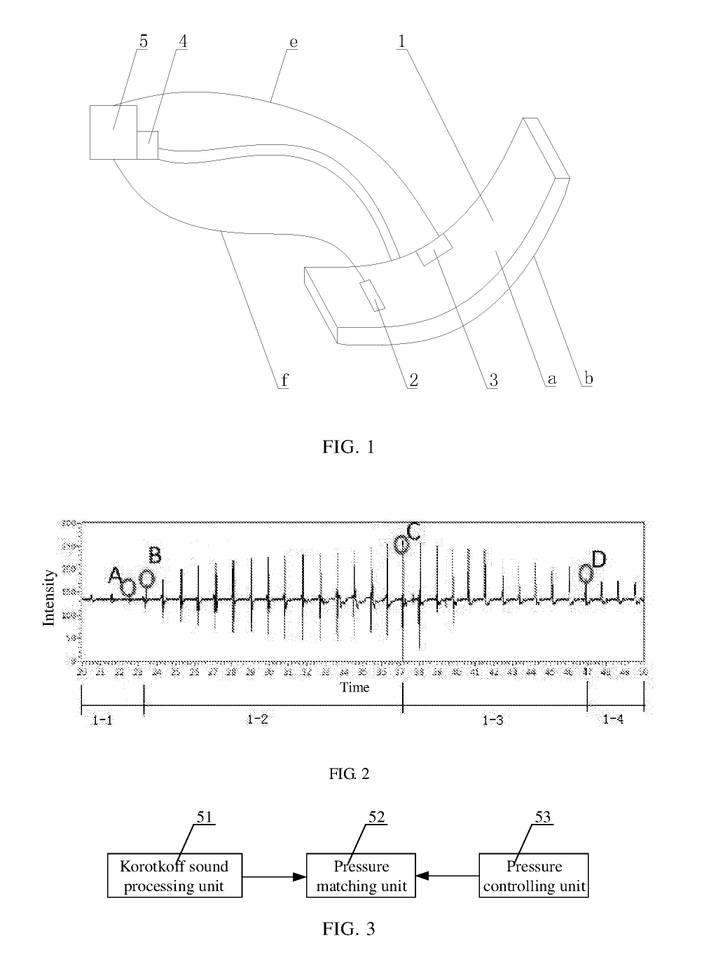

An embodiment of the present disclosure provides a blood pressure measurement instrument, as illustrated in FIG. 1, including a cuff 1 capable of being worn at a measured position on a body, a pressure sensor 2, a sound sensor 3, a pressure regulator 4 and a processor 5.

An inner ring a of the cuff 1 is wound at the measured position, such as an arm, and the pressure regulator 4 is connected with the cuff 1 and is configured to regulate a pressure applied to the measured position by the cuff 1. The pressure sensor 2 is arranged on the cuff 1, and is configured to acquire the pressure applied to the measured position by the cuff 1 during a period of time when the pressure is changed. The sound sensor 3 is positioned between the cuff 1 and the measured position in the measuring process , such as on the inner ring a of the cuff 1 as illustrated in FIG. 1, and is configured to acquire blood flow sound at the measured position. According to an embodiment of the present disclosure, the blood flow sound includes Korotkoff sound. Certainly, those skilled in the art may understand that a blood pressure value can also be determined according to other parameter values of the detected blood flow sound. The present disclosure describes the technical solution by taking the Korotkoff sound as an example. The processor 5 is connected with the pressure sensor 2 and the sound sensor 3, and is configured to acquire an appearance moment and a disappearance moment of the Korotkoff sound according to acquisition results of the pressure sensor 2 and the sound sensor 3 during the period of time when the pressure is changed so as to determine a blood pressure value. The processor 5 may be a special processor (for example, a Field Programmable Gate Array (FPGA), a Micro Controller Unit (MCU) and the like) or a universal processor (for example, a Central Processing Unit (CPU).

In an embodiment of the present disclosure, when the cuff 1 is worn on the arm, the inner ring a of the cuff 1 is in contact with the arm, an outer ring b of the cuff 1 faces the outside, and the position of the cuff 1 is regulated to enable the sound sensor positioned on the inner ring a of the cuff 1 to be attached to the brachial artery position of the fossa cubitalis, so that the sound sensor 3 can be clung to the brachial artery to acquire the blood flow sound, and meanwhile, due to the barrier action of the cuff 1, acquisition on other noise outsides can be avoided to the greatest extent, so that acquired sound signals are clear and stable.

In an embodiment of the present disclosure, as illustrated in FIG. 2, the sound sensor 3 is arranged at the artery at the measured position of the body and can always acquire the blood flow sound in the vessel. The blood flow sound acquired by the sound sensor will be analyzed below.

In an embodiment of the present disclosure, during a stage 1-1 in FIG. 2, the Korotkoff sound is not generated at the moment, the blood flow sound only includes pulse sound generated by pulse beats when blood flows in a pulse. The frequency and the amplitude of the pulse sound are basically unchanged. A changeable pressure is applied to the measured position; after the artery vessel blocks up blood flow via external force compression, decompression is gradually carried out; along with reduction of the pressure, the blocked blood flow in the artery vessel can break through the vessel again at a certain moment; and when the blood flow breaks through the vessel again, the Korotkoff sound appears. At the appearance moment of the Korotkoff sound, a value of the pressure correspondingly applied to the measured position is a measured systolic blood pressure.

The Korotkoff sound proceeds to a stage 1-2 at the appearance moment; during the stage 1-2, the Korotkoff sound is generated and reaches a maximum value; and the Korotkoff sound is a series of sound signals which gradually become strong from appearance and then are gradually weakened to disappear, and a frequency of the Korotkoff sound is the same with that of the pulse sound, and thus, the blood flow sound during the stage 1-2 is sound formed after the pulse sound and the Korotkoff sound are overlaid.

When the Korotkoff sound is gradually increased to the maximum value, i.e., the Korotkoff sound proceeds to a stage 1-3 as illustrated in FIG. 2, the blood flow sound during the stage 1-3 still is the sound formed after the pulse sound and the Korotkoff sound are overlaid, and the Korotkoff sound is gradually weakened during the stage 1-3. When the Korotkoff sound disappears, the value of the pressure which corresponds to the disappearance moment of the Korotkoff sound and is applied to the measured position is referred to as a measured diastolic blood pressure.

From the disappearance moment of the Korotkoff sound, the Korotkoff sound proceeds to a stage 1-4; during the stage 1-4, the Korotkoff sound has disappeared, and thus, the blood flow sound during such stage only includes the pulse sound; and at the moment, the pressure applied to the measured position is completely released in an accelerating mode so as to complete measurement on a blood pressure.

As illustrated in FIG. 2, the value of the blood flow sound at the measured position, which is acquired by the blood pressure measurement instrument of the embodiment, means an intensity peak value of the pulse sound in each pulse period or an intensity peak value of the blood flow sound formed by overlaying of the pulse sound and the Korotkoff sound. Signal amplitudes of the blood flow sound during the stage 1-2 and during the stage 1-3 are obviously greater than that of the blood flow sound during the stage 1-1, and during the stage 1-2 and during the stage 1-3, the acquisition result of the sound sensor 3 is the blood flow sound formed after the Korotkoff sound and the pulse sound are overlaid, and the Korotkoff sound and the pulse sound in the blood flow sound cannot be separately measured, and thus, in order to facilitate illustration, the blood flow sound measured during the stages 1-2 to 1-3 is directly called as the Korotkoff sound below.

Generally, the artery position, which is relatively thin in the fat layer on a human body, needs to be selected for blood pressure measurement, which is convenient to apply the pressure to the measured position and meanwhile, can also acquire the clear sound signals at the measured position; generally, arteries at joints, i.e., the brachial artery inside an elbow joint of an upper arm, a femoral artery at a leg end, a radial artery inside a wrist and the like, can be selected to measure the blood pressure; and in the embodiment, in order to facilitate measurement, illustration is carried out by taking the brachial artery inside the elbow joint of the upper arm as an example.

According to different genders and age groups of measured people, ranges of blood pressure normal values are also correspondingly different, and by taking males at the age of 51 to 55 as an example, the range of the blood pressure normal values is that the systolic blood pressure is 120 to 140 mmHg, and the diastolic blood pressure is 75 to 95 mmHg.

In an embodiment of the present disclosure, as illustrated in FIG. 1, the pressure regulator 4 is connected with the cuff 1, pressurization or decompression is carried out on the inside of the cuff 1 under a certain intensity, the pressure applied to the measured position of the body by the cuff 1 is regulated to uniformly reduce intensity of an external force after providing the uniform external force to compress and block up the blood flow, so that the blocked blood flow breaks through the vessel again at a certain moment to be recovered to flow.

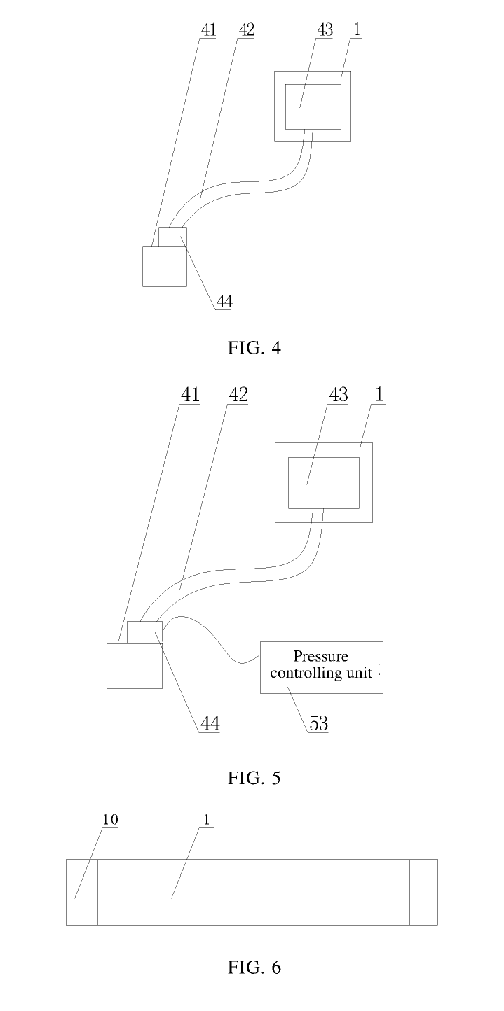

In an embodiment of the present disclosure, as illustrated in FIG. 4, the pressure regulator 4 includes an air pump 41, an air delivery pipe 42 connected with the air pump 41 and an airbag 43 connected to the other end of the air delivery pipe 42, the airbag 43 is arranged inside the cuff 1, and the air pump 41 inflates and deflates the airbag 43 by the air delivery pipe 42.

By using the pressure regulator 4 consisting of the air pump 41, the air delivery pipe 42 and the airbag 43, when the air pump 41 inflates the airbag 43 by the air delivery pipe 42, the airbag 43 arranged in the cuff 1 is uniformly expanded around the periphery of the arm, so that the brachial artery bound by the inner ring a of the cuff 1 is uniformly squeezed to gradually block off the blood flow; and when the air pump 41 gradually releases air in the airbag 43 by the air delivery pipe 42, the airbag 43 is uniformly shrunk around the periphery of the arm, so that a squeezing force applied to the brachial artery bound by the inner ring a of the cuff 1 is gradually reduced until the blood flow in the brachial artery breaks through obstruction at a certain moment to break through the vessel again to be recovered to flow. Therefore, the pressure applied to the periphery of the arm can be guaranteed to be relatively uniform, and a blood pressure measurement error caused by an uneven partial stress on the arm is avoided.

Moreover, the pressure regulator 4 can also enable an elastic band wound at the brachial artery of the arm to be gradually shrunk to apply a tightening force to the arm by pulling the cuff with the elastic band, which is wound at the brachial artery of the arm, and gradually loosen the elastic band to release the tightening force applied to the arm when the tightening force is sufficiently large so as to carry out measurement on the blood pressure at the brachial artery, and the generation mode of the pressure is not specifically defined herein, as long as the pressure can be gradually increased or reduced under the uniform intensity and the pressure is applied to the measured position on the body by the cuff 1 for temporarily blocking off the blood flow.

In an embodiment of the present disclosure, as illustrated in FIG. 6, the cuff 1 further includes connecting pieces 10 arranged at both ends of the cuff 1, and when the cuff 1 is wound at the measured position, the connecting pieces 10 are used for fixing both ends of the cuff 1.

In the blood pressure measuring process, in order to ensure that the airbag 43 stably applies or releases the pressure to the arm in the pressurizing or decompressing process and avoid the blood pressure measurement failure caused by falling or shifting of the cuff 1 in the pressurizing process, after the cuff 1 is wound at a to-be-measured position on the arm, the cuff 1 is fixed firmly on the arm by the connecting pieces at both ends of the cuff 1, and after measurement is completed, the connecting pieces 10 for fixing the cuff 1 are opened, and the cuff 1 is taken down.

According to an embodiment of the present disclosure, the connecting pieces 10 may be overlapping buckle structures which are arranged telescopically and can be mutually fixed, may be bandages, and may also be adhering buckles. In an embodiment of the present disclosure, the connecting pieces 10 are adhering buckle structures. When the adhering buckle structures are used, in one aspect, the connecting pieces 10 can be adhered firmly only by pressing both ends with adhering structures in an overlapping mode in the using process, and the connecting pieces 10 can be torn off with one single hand when being taken down, and can be repeatedly utilized; and in the other aspect, the adhering buckle structures are not limited to a fixed position, overlaps of both the ends with the adhering structures are changeable in size so as to facilitate regulating an adhering position according to different thicknesses of arms of users, and thus, when the cuff 1 is guaranteed to be firmly fixed, the blood pressure measurement instrument also has relatively high use comfort.

The blood pressure measurement instrument of the embodiments of the present disclosure includes the cuff capable of being worn at the measured position on the body, the pressure sensor, the sound sensor, the pressure regulator and the processor.

The pressure regulator is connected with the cuff, and is configured to regulate the pressure applied to the measured position by the cuff. The pressure sensor is arranged on the cuff, and is configured to acquire the pressure applied to the measured position by the cuff during the period of time when the pressure is changed. The sound sensor is positioned between the cuff and the measured position in the measuring process and is configured to acquire the blood flow sound at the measured position, and the blood flow sound includes the Korotkoff sound.

The processor is communicated with the pressure sensor and the sound sensor, and is configured to acquire the pressure values respectively corresponding to the appearance moment and the disappearance moment of the Korotkoff sound according to the acquisition results of the pressure sensor and the sound sensor during the period of time when the pressure is changed so as to determine the blood pressure value.

The pressure is applied to the cuff worn at the measured position on the body or released by the pressure regulator, so that the measured position is applied with the changeable pressure from the cuff; the pressure sensor acquires the value of the pressure applied to the measured position by the cuff; the sound sensor acquires the Korotkoff sound at the measured position; in the processor, an appearance point and a disappearance point of the Korotkoff sound are compared and determined; and the pressure values corresponding to the appearance point and the disappearance point of the Korotkoff sound are output as the systolic blood pressure and the diastolic blood pressure obtained by measurement. Influence of subjective judgment of an operator on a measured value is avoided, and accuracy of a measurement result is improved.

According to the embodiment as illustrated in FIG. 1, the processor is connected with the pressure sensor 2 by a data line f to receive pressure data acquired by the pressure sensor 2, is connected with the sound sensor 3 by a data line/cable e to receive and process blood flow sound data acquired by the sound sensor 3, and obtains a blood pressure value of a measured person after matching the appearance moment and the disappearance moment of the Korotkoff sound, which are acquired, with the corresponding pressure data acquired by the pressure sensor 2. The data acquiring and processing process of the processor will be illustrated below.

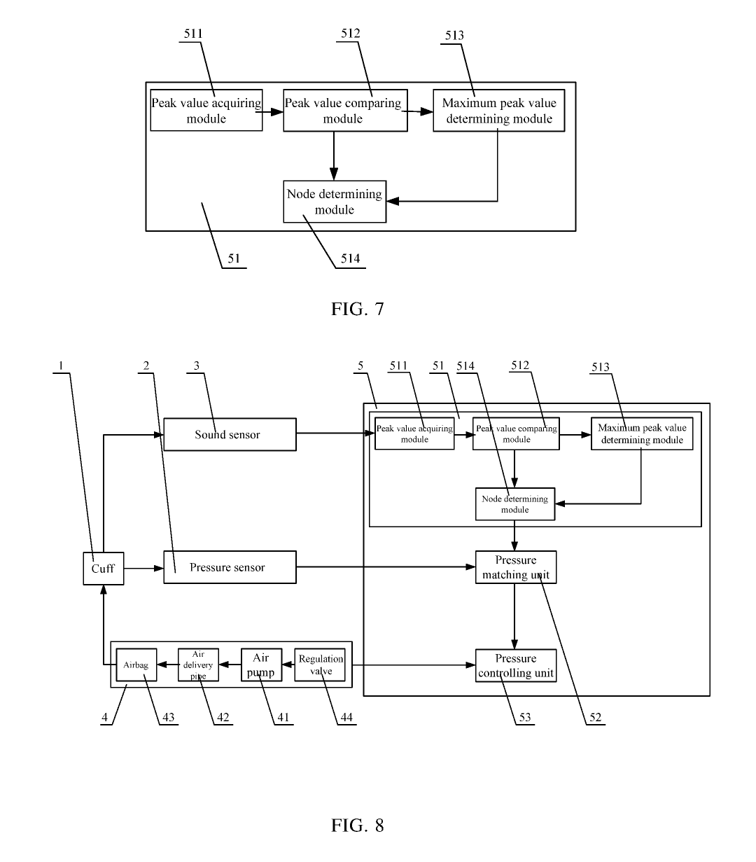

According to an embodiment of the present disclosure, as illustrated in FIG. 3, the processor 5 includes a Korotkoff sound processing unit 51. The Korotkoff sound processing unit 51 is configured to carry out comparison on the sound signals collected by the sound sensor 3 and determine the appearance moment and the disappearance moment of the Korotkoff sound. As illustrated in FIG. 7, the Korotkoff sound processing unit 51 includes a peak value acquiring module 511, a peak value comparing module 512, a maximum peak value determining module 513 and a node determining module 514.

The peak value acquiring module 511 is configured to acquire a sound intensity peak value of the Korotkoff sound at the measured position in each period. As illustrated in FIG. 2, two adjacent wave peaks are one sound intensity period, and points A, B, C and D in FIG. 2 are all sound intensity peak values in the period.

The peak value comparing module 512 is connected with the peak value acquiring module 511, and is configured to, according to acquisition results of the peak value acquiring module 511, compare a peak value in a current period with a peak value in a previous period.

By taking FIG. 2 as an example, at a moment as illustrated at the point B, a peak value of the B point is the peak value of the current period, a peak value of the point A is the peak value of the previous period, and at the moment, the peak value corresponding to the point B is compared with the peak value corresponding to the point A. When the operation is carried out to a next period, a peak value in one period after the point B is used as the peak value of the current period, a peak value corresponding to the point B is changed into the peak value of the previous period, and then the peak value in one period after the point B is compared with the peak value corresponding to the point B.

The maximum peak value determining module 513 is connected with the peak value comparing module 512, and is configured to, when the peak value comparing module 512 concludes that the peak value in the current period is smaller than the peak value in the previous period for the first time, determine the peak value in the previous period as a maximum peak value of the Korotkoff sound; the period in which the maximum peak value of the Korotkoff sound is determined is referred to as a reference period.

For example, the Korotkoff sound is a series of sound which gradually becomes strong from appearance and is gradually weakened to disappear after reaching a maximum value, and in the peak value comparing module 512, the peak value in the current period is always compared with the peak value in the previous period. When the Korotkoff sound is during a stage of gradually becoming strong since appearing, the peak value in the current period is always greater than the peak value in the previous period. When the Korotkoff sound reaches the maximum value, the Korotkoff sound proceeds to a stage of being gradually weakened, and then in next comparison, the peak value in the current period is always smaller than the peak value in the previous period. Therefore, when the peak value in the current period is smaller than the peak value in the previous period for the first time, the peak value in the previous period is determined as the maximum value of the Korotkoff sound by the maximum peak value determining module 513.

By taking FIG. 2 as the example, when the current period is a period in which the point C is located, a peak value corresponding to the point C is compared with a peak value in one period before the point C, and the peak value corresponding to the point C is greater than the peak value of the one period before the point C; and then the current period is changed into one period after the point C, a peak value in the one period after the point C is compared with the peak value corresponding to the point C, the comparison result is that the peak value in the one period after the point C is smaller than the peak value corresponding to the point C, and at the moment, the peak value in the current period is smaller than the peak value in the previous period for the first time, so the peak value corresponding to the point C is determined as the maximum peak value of the Korotkoff sound and meanwhile, the period in which the point C is located is referred to as the reference period.

The node determining module 514 is connected with the peak value comparing module 512 and configured to, when the peak value comparing module 512 concludes that the peak value in the current period is greater than or equal to 105% to 110% of the peak value in the previous period for the first time, determine a moment when the peak value in the current period appears as the appearance moment of the Korotkoff sound. By taking FIG. 2 as the example, during the stage 1-1, the peak value in each current period is compared with the peak value in its previous period and is basically the same in size, and when the current period is a period in which the point B is located, the peak value corresponding to the point B is compared with the peak value corresponding to the point A, the peak value corresponding to the point B is greater than 105% to 110% of the peak value corresponding to the point A, and then the point B is determined as the appearance moment of the Korotkoff sound in the node determining module 514.

When a threshold interval for determining the appearance moment of the Korotkoff sound is set to be smaller than 105%, due to a small increasing proportion, small-range value numerical fluctuation generated by interference of other noise may be determined as the appearance moment of the Korotkoff sound in the node determining module 514, resulting in a measurement error; and if the threshold interval is set to be greater than 110%, the peak value comparing module 512 may be insensitive to the comparison result so as to cause delay of determining time of the node determining module 514 on the appearance moment of the Korotkoff sound, thereby causing inaccuracy of a measured value.

Optionally, the node determining module 514 is further connected with the maximum peak value determining module 513, and is configured to, when the peak value comparing module 512 compares a peak value in each period after the reference period with the maximum peak value of the Korotkoff sound until the peak value comparing module 512 obtains one period in which a peak value of this one period is smaller than or equal to 65% to 75% of the maximum peak value of the Korotkoff sound, determine a moment when the peak value in the one period appears as the disappearance moment of the Korotkoff sound. For example, during the stage 1-3 in FIG. 2, at the moment, the peak value corresponding to the point C has been determined as the maximum peak value of the Korotkoff sound; during the stage 1-3, the peak value in each current period is compared with the peak value corresponding to the point C, until the current period is a period in which the point D is located, a peak value corresponding to the point D is compared with the peak value corresponding to the point C, the peak value corresponding to the point D is smaller than or equal to 65% to 75% of the peak value corresponding to the point C, and at the moment, the point D is determined as the disappearance moment of the Korotkoff sound.

If a threshold interval for determining the disappearance moment of the Korotkoff sound is set to be greater than 75%, the case of determining the relatively weak Korotkoff sound in a later period as disappearance too early in the node determining module 514 due to occasional small-range numerical fluctuation may be caused; and if the threshold interval is set to be smaller than 65%, a case that the node determining module 514 is difficult to determine the disappearance moment of the Korotkoff sound or a large error is generated may be caused.

In an embodiment of the present disclosure, as illustrated in FIG. 3, the processor 5 further includes a pressure matching unit 52. As illustrated in FIG. 8, the pressure matching unit 52 is connected with the Korotkoff sound processing unit 51 and the pressure sensor 2, and is configured to receive the appearance moment and the disappearance moment of the Korotkoff sound, which are output by the Korotkoff sound processing unit 51, carry out matching on a pressure value acquired by the pressure sensor 2 at the appearance moment of the Korotkoff sound to use the pressure value a systolic blood pressure and carry out matching on a pressure value acquired by the pressure sensor 2 at the disappearance moment of the Korotkoff sound to use the pressure value as a diastolic blood pressure.

The processor 5 acquires the appearance moment and the disappearance moment of the Korotkoff sound during the period of time when the pressure is changed and respectively corresponds to the pressure values at the corresponding moments. As illustrated in FIG. 3, the processor 5 further includes a pressure controlling unit 53. As illustrated in FIG. 5, in order to enable the pressure to be slowly and uniformly changed, the pressure regulator 4 further includes a regulation valve 44 mounted on the air pump 41.

According to an embodiment of the present disclosure, as illustrated in FIG. 8, the pressure controlling unit 53, by controlling the regulation valve 44 of the pressure regulator 4, enables the air pump 43 to firstly pressurize the airbag 43 in the cuff 1 at a speed of 2 to 4 mmHg/s by the air delivery pipe 42.

If the pressurizing speed is smaller than 2 mmHg/s, pressurization time may be excessively long, so that time of blood pressure measurement is prolonged; and if the pressurizing speed is greater than 4 mmHg/s, the arm of the measured person may feel pain or other uncomfortable feelings may be caused due to the excessively high pressurizing speed.

According to a pressure signal acquired by the pressure sensor 2, when an air pressure in the airbag 43 reaches 210 mmHg to 260 mmHg, the pressure controlling unit 53, by controlling the regulation valve 44 of the pressure regulator 4, enables the air pump 43 to release the pressure of the airbag 43 in the cuff 1 at a speed of 2 to 4 mmHg/s by the air delivery pipe 42.

If a maximum value of the air pressure in the airbag 43 is set to be smaller than 210 mmHg, inaccuracy of measuring the systolic blood pressures of some critical patients with high blood pressures may be caused due to an insufficient pressurization peak value; and if the maximum value of the air pressure in the airbag 43 is set to be greater than 260 mmHg, due to the excessive pressurization of the airbag 43, the arm of the measured person may feel pain on the pressure to bring pain to the measured person.

If a decompression speed is smaller than 2 mmHg/s, decompression time may be excessively long, so that time of blood pressure measurement is prolonged; and if the decompression speed is greater than 4 mmHg/s, due to the excessively high decompression speed, inaccuracy of acquisition of the sound sensor 3 on the Korotkoff sound and in the pressure matching unit 52 of the processor 5, error generation in the process of carrying out matching on the appearance moment and the disappearance moment of the Korotkoff sound and the pressure values measured by the pressure sensor 2 may be caused.

In two to three seconds after the processor 5 determines the disappearance moment of the Korotkoff sound, at the moment, measurement of the blood pressure measurement instrument on the blood pressure has been finished, and the measured value has been generated, and thus, at the moment, the pressure controlling unit 53, by controlling the regulation valve 44 of the pressure regulator 4, enables the air pump to improve a pressure releasing speed in the airbag 43 and release the pressure in the airbag 43 at a speed of 8 to 12 mmHg/s until the pressure in the airbag 43 is completely released.

If the decompression speed is smaller than 8 mmHg/s, due to the excessively low decompression speed, the measurement time may be prolonged; and if the decompression speed is greater than 12 mmHg/s, due to the excessively high decompression speed, discomfort may be caused to the arm of the measured person.

In an embodiment of the present disclosure, the processor 5 in the blood pressure measurement instrument of the present disclosure adopts a 32-bit ARM processor. The 32-bit ARM processor is stable to work and low in power consumption, so that when functions of the processor 5 are achieved, energy consumption is reduced.

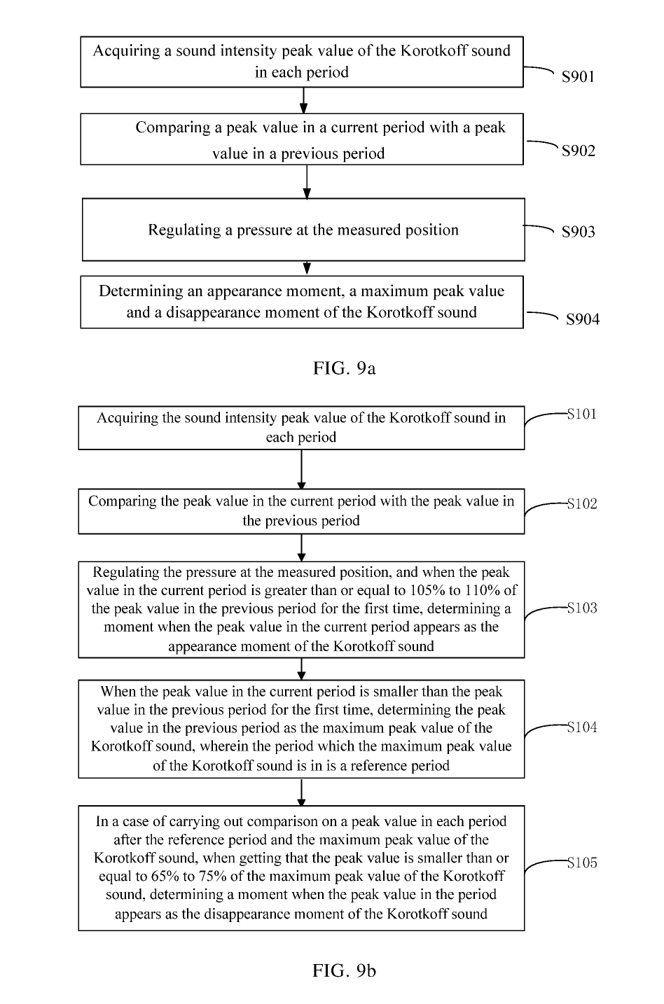

The present disclosure provides a Korotkoff sound identifying method for acquiring blood flow sound at a measured position, the blood flow sound including Korotkoff sound. As illustrated in FIG. 9a, according to an embodiment of the present disclosure, the method may include the following operations:

S901: acquiring a sound intensity peak value of the Korotkoff sound in each period.

S902: comparing a peak value in a current period with a peak value in a previous period.

S903: regulating a pressure at the measured position.

S904: determining an appearance moment, a maximum peak value and a disappearance moment of the Korotkoff sound.

According to another example of the present disclosure, as illustrated in FIG. 9b, the method may further include the following operations:

S101: acquiring the sound intensity peak value of the Korotkoff sound in each period.

According to an embodiment of the present disclosure, as illustrated in FIG. 2, a high-site signal in each period is the sound intensity peak value in the period. The Korotkoff sound is a sound signal with the same frequency period with pulse sound, there is one sound intensity peak value the in each period, and the Korotkoff sound can gradually become strong after being generated, and is gradually weakened to disappear after reaching a maximum value. Before the Korotkoff sound appears, i.e., during the stage 1-1 in FIG. 2, the sound intensity peak value is only a pulse sound signal peak value generated when the pulse beats; at the appearance moment of the Korotkoff sound, the Korotkoff sound proceeds to the stage 1-2; during the stage 1-2, the Korotkoff sound is generated and gradually becomes strong; when reaching the maximum value, the Korotkoff sound proceeds to the stage 1-3; during the stage 1-3, the Korotkoff sound is gradually weakened from the maximum value; and both the sound intensity peak values during the stages 1-2 to 1-3 are sound peak values formed after the Korotkoff sound and the pulse sound are overlapped. From the disappearance moment of the Korotkoff sound, the Korotkoff sound proceeds to the stage 1-4; and during the stage 1-4, the Korotkoff sound disappears, and the sound intensity peak value is only the pulse sound signal peak value generated when the pulse beats.

S102: comparing the peak value in the current period with the peak value in the previous period.

According to one example of the present disclosure, every when the sound intensity peak value of the Korotkoff sound in one period is acquired, the peak value in the current period is compared with the peak value in the previous period. As illustrated in FIG. 2, when the current period is the period in which the point B is located, a sound intensity peak value corresponding to the point B is compared with a sound intensity peak value corresponding to the point A. When the operation is carried out to a next period, the current period is a period after the period in which the point B is located, and at the moment, a sound intensity peak value of the period after the period in which the point B is located is compared with the sound intensity peak value in the period in which the point B is located.

S103: regulating the pressure at the measured position, and when the peak value in the current period is greater than or equal to 105% to 110% of the peak value in the previous period for the first time, determining a moment when the peak value in the current period appears as the appearance moment of the Korotkoff sound.

According to an embodiment of the present disclosure, the Korotkoff sound is sound generated due to friction and collision between the blood flow and the vessel in a period of time when a pressure applied externally is gradually reduced after forming temporary blocking on the artery vessel until the blood flow breaks through the vessel again at a certain moment to be recovered to flow, and thus, the pressure at the measured position needs to be regulated, and until the blood flow can break through the vessel again, the Korotkoff sound is generated. As illustrated in FIG. 2, when the current period is the period in which the point B is located, the sound intensity peak value corresponding to the point B is compared with the sound intensity peak value corresponding to the point A. the sound intensity peak value corresponding to the point B is greater than 105% to 110% of the sound intensity peak value corresponding to the point A, and thus, the point B is determined as the appearance moment of the Korotkoff sound.

If the peak value in the current period is greater than or equal to the peak value in the previous period by smaller than 105%, a single error may be generated due to influence of other noise; and if the peak value in the current period is greater than or equal to the peak value in the previous period by over 110%, delay of determining time of the appearance moment of the Korotkoff sound may be caused due to measurement insensitivity.

S104: when the peak value in the current period is smaller than the peak value in the previous period for the first time, determining the peak value in the previous period as the maximum peak value of the Korotkoff sound, wherein the period in which the maximum peak value of the Korotkoff sound is determined is referred to as a reference period.

According to an embodiment of the present disclosure, as illustrated in FIG. 2, after the appearance moment of the Korotkoff sound, the Korotkoff sound proceeds to the stages 1-2 to 1-3; during the stage 1-2, the Korotkoff sound gradually becomes strong from generation, and proceeds to the stage 1-3 after the maximum peak value of the Korotkoff sound is reached; the Korotkoff sound is gradually weakened to disappear; the maximum peak value of the Korotkoff sound is judged, i.e., the peak value in each current period during the stages 1-2 to 1-3 is compared with the peak value in the previous period; the peak value in the current period during the stage 1-2 is greater than the peak value in the previous period, which illustrates that the Korotkoff sound still gradually becomes strong; when the current period is a period in which the point C is located, a peak value corresponding to the point C is compared with a peak value in one period before the point C; if the peak value corresponding to the point C is still greater than the peak value in the one period before the point C, the current period proceeds to a next period of the point C; at the moment, a peak value in the next period of the point C is compared with the peak value corresponding to the point C; and if the peak value in the next period of the point C is smaller than the peak value corresponding to the point C, the peak value corresponding to the point C is determined as the maximum peak value of the Korotkoff sound, and the period in which the point C is determined is referred to as the reference period.

S105: in a case of carrying out comparison on a peak value in each period after the reference period and the maximum peak value of the Korotkoff sound, when getting that the peak value is smaller than or equal to 65% to 75% of the maximum peak value of the Korotkoff sound, determining a moment when the peak value in the one period appears as the disappearance moment of the Korotkoff sound.

According to an embodiment of the present disclosure, after the reference period, as illustrated in FIG. 2, the Korotkoff sound proceeds to the stage 1-3; and after the maximum peak value of the Korotkoff sound appears, the Korotkoff sound starts to be gradually weakened to disappear. During the stage 1-3, comparison on the peak value is that: the peak value in each current period is compared with the maximum peak value of the Korotkoff sound, which is determined in the step S104, a ratio of the peak value in the current period to the maximum peak value (the peak value corresponding to the point C) of the Korotkoff sound is calculated, when the current period is a period in which the point D in FIG. 2 is located, a peak value corresponding to the point D is compared with the peak value corresponding to the point C, and when the peak value corresponding to the point D is smaller than 65% to 75% of the peak value corresponding to the point C, the point D is determined as the disappearance moment of the Korotkoff sound.

When the peak value in the current period is smaller than or equal to the maximum peak value of the Korotkoff sound by over 75%, it is illustrated that the Korotkoff sound is weakened, but the Korotkoff sound still exists; and the pulse sound in the blood flow sound always exists and is kept having a certain intensity, and thus, the case that the peak value in the current period is smaller than or equal to the maximum peak value of the Korotkoff sound by smaller than 65% is generally caused by an error generated in a sound period or sudden disappearance of other noise, and if a judgment standard on the disappearance moment of the Korotkoff sound is set as a standard that the peak value in the current period is smaller than or equal to the maximum peak value of the Korotkoff sound by smaller than 65%, it is possibly difficult to measure the disappearance moment of the Korotkoff sound.

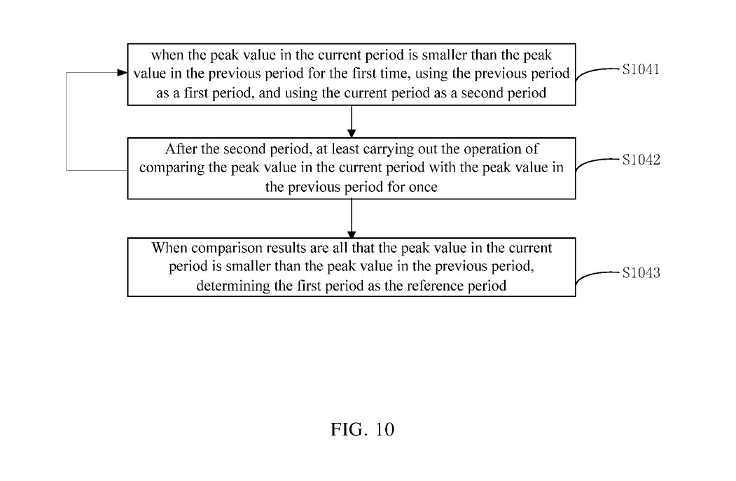

In an embodiment of the present disclosure, as illustrated in FIG. 10, the step S104 of the Korotkoff sound identifying method further includes the following operations:

S1041: when the peak value in the current period is smaller than the peak value in the previous period for the first time, referring to the previous period as a first period, and referring to the current period as a second period.

S1042: after the second period, at least carrying out an operation of comparing a peak value in a new current period with a peak value in a new previous period.

S1043: when a comparison result of the above operation each shows that the peak value in the new current period is smaller than the peak value in the new previous period, determining the first period as the reference period.

According to an embodiment of the present disclosure, in the step S104 of determining the maximum peak value of the Korotkoff sound and the period in which the maximum peak value is determined, in order to avoid a determination fault caused by the single error, as illustrated in FIG. 2, if the current period is one period after the period in which the point C is located, in step S1041, the peak value in one period after the period in which the point C is located is compared with the peak value in the period in which the point C is located, it is obtained by comparison that the peak value in one period after the period in which the point C is located is smaller than the peak value in the period in which the point C is located, and at the moment, the period in which the point C is located is denoted as the first period, and the current period, i.e., one period after the period in which the point C is located, is denoted as the second period.

The current period is advanced along a direction of time increasing in FIG. 2; at the moment, the current period is two periods after the period in which the point C is located; in step S1042, the peak value in the current period (two periods after the period in which the point C is located) is compared with the peak value in the previous period (one period after the period in which the point C is located); if a comparison result is also that the peak value in the current period is smaller than the peak value in the previous period, comparison in S1041 is correct; then the step S1043 is carried out; and when the a comparison result of the above operation each shows that the peak value in the new current period is smaller than the peak value in the new previous period, the first period is determined as the reference period. If the comparison result is that the peak value in two periods after the period in which the point C is located is greater than the peak value in one period after the period in which the point C is located, a comparison result is different from that in step S1041, and then it is considered that the comparison result in the step S1041 is a measurement error caused by various external reasons; at the moment, the Korotkoff sound is still during the stage 1-2 in FIG. 2, and then operation is jumped back to the step S1041; and after the current period is advanced along the direction of time increasing in FIG. 2, comparison is carried out again.

In an embodiment of the present disclosure, the step S1042 is carried out for once after the step S1041; generally, the step S1042 is at least carried out for once after the step 1041, and repeated verification can also be carried out as required; and if the operation is jumped to step S1041 after comparison in the step S1042, when the operation is carried out to the step S1042 via the step S1041 again after the current period is advanced, comparison still needs to be carried out again for at least once, until the next step S1043 is carried out after the comparison results in steps S1042 and S1041 are consistent.

In an embodiment of the present disclosure, before the appearance moment of the Korotkoff sound, regulating the pressure at the measured position includes the following operation:

S106: pressurizing the measured position at a speed of 2 to 4 mmHg/s; and when the pressure at the measured position reaches 210 mmHg to 260 mmHg, releasing the pressure at the measured position at a speed of 2 to 4 mmHg/s;

In two to three seconds after the disappearance moment of the Korotkoff sound, regulating the pressure at the measured position further includes the operation: S107, releasing the pressure at the measured position at a speed of 8 to 12 mmHg/s.

According to an embodiment of the present disclosure, when the steps S101 to S103 are carried out, the step S106 is carried out for matching to regulate the pressure at the measured position, i.e., firstly, the measured position is gradually pressurized at the speed of 2 to 4 mmHg/s, artery blood flow at the measured position is gradually blocked off in the pressure increasing process, the pressure at the measured position is released at the speed of 2 to 4 mmHg/s when reaching 210 mmHg to 260 mmHg, the blood flow breaks through the blocking to reflow at a certain moment when the pressure is gradually reduced, the Korotkoff sound is generated, and at the moment, by comparison in step S103, the appearance moment of the Korotkoff sound is determined and pressurization and pressure release are carried out uniformly and slowly, so that identification accuracy on the appearance moment and the disappearance moment of the Korotkoff sound is ensured.

In the step S106, both the pressure increasing and releasing processes are carried out at the speed of 2 to 4 mmHg/s, and if pressure increasing or releasing speeds are greater than 4 mmHg/s, a span between two comparisons in S103 is excessively large, resulting in poor identification accuracy on the Korotkoff sound; and if the pressure increasing or releasing speeds are smaller than 2 mmHg/s, pressure increasing and releasing time can be excessively long, so that ineffective working time in the Korotkoff sound identification is increased. When the pressure at the measured position reaches 210 mmHg to 260 mmHg, the blood flow at the measured position is necessarily blocked off by the pressure, and at the moment, the pressure at the measured position is gradually released, so that in one aspect, generation of the Korotkoff sound is ensured, and in the other aspect, working time before the Korotkoff sound is generated is also shortened.

After the step S105 is completed, S107 is carried out, and in 2 to 3 s after the disappearance moment of the Korotkoff sound, the pressure at the measured position is released at the speed of 8 to 12 mmHg/s. At the moment, the step S103 of identifying and determining the appearance moment of the Korotkoff sound and the step S105 of identifying and determining the disappearance moment of the Korotkoff sound have been completed, and the pressure at the measured position is rapidly released at the speed of 8 to 12 mmHg/s until the pressure at the measured position is completely released. At the moment, if the releasing speed of the pressure is smaller than 8 mmHg/s, the ineffective working time can be prolonged, and if the releasing speed of the pressure is greater than 12 mmHg/s, discomfort at the measured position can be caused.

An embodiment of the present disclosure provides a Korotkoff sound identification apparatus. As illustrated in FIG. 7, identification is carried out on Korotkoff sound by using the Korotkoff sound identifying method. The Korotkoff sound identification apparatus includes a Korotkoff sound processing unit 51. The Korotkoff sound processing unit 51 includes a peak value acquiring module 511, a peak value comparing module 512, a maximum peak value determining module 513 and a node determining module 514.

The peak value acquiring module 511 is configured to acquire a peak value of sound intensity of the Korotkoff sound at a measured position in each period.

The peak value comparing module 512 is connected with the peak value acquiring module, and is configured to compare a peak value in a current period with a peak value in a previous period according to an acquisition result of the peak value acquiring module.

The maximum peak value determining module 513 is connected with the peak value comparing module 512, and is configured to determine a maximum peak value of the Korotkoff sound. For example, the peak value comparing module 512 concludes that the peak value in the current period is smaller than the peak value in the previous period for the first time, the peak value in the previous period is determined as the maximum peak value of the Korotkoff sound,; the period in which the maximum peak value of the Korotkoff sound is determined is used as a reference period.

The node determining module 514 is connected with the peak value comparing module 512, and is configured to determine an appearance moment and a disappearance moment of the Korotkoff sound. For example, when the peak value comparing module 512 concludes that the peak value in the current period is greater than or equal to 105% to 110% of the peak value in the previous period for the first time, a moment when the peak value in the current period appears is determined as the appearance moment of the Korotkoff sound.

In an embodiment of the present disclosure, the node determining module 514 is further connected with the maximum peak value determining module 513, and is configured to, when the peak value comparing module 512 compares a peak value in each period after the reference period with the maximum peak value of the Korotkoff sound until the peak value comparing module 512 obtains one period in which a peak value of this one period is smaller than or equal to 65% to 75% of the maximum peak value of the Korotkoff sound, determine a moment when the peak value in the one period appears as the disappearance moment of the Korotkoff sound.

In an embodiment of the present disclosure, the Korotkoff sound identification apparatus further includes a pressure controlling unit. The pressure controlling unit is configured to: before the appearance moment of the Korotkoff sound, pressurize the measured position at a speed of 2 to 4 mmHg/s; when a pressure at the measured position reaches 210 mmHg to 260 mmHg, release the pressure at the measured position at a speed of 2 to 4 mmHg/s; and in two to three seconds after the disappearance moment of the Korotkoff sound, release the pressure at the measured position at a speed of 8 to 12 mmHg/s.

The foregoing embodiments merely are exemplary embodiments of the present disclosure, but the scope of the present disclosure is not limited thereto; those skilled in the art may easily think of variations or improvements in the technical scope disclosed by the embodiments of the present disclosure, and those variations and improvements shall fall within the scope of the present disclosure. Therefore, the scope of the present disclosure shall be determined by the scope of the claims.

The application claims priority to the Chinese patent application No. 201610317930.9, filed May 12, 2016, the entire disclosure of which is incorporated herein by reference as part of the present application.

1. A blood pressure measurement instrument, comprising: a cuff capable of being worn at a measured position on a body of a user, a pressure sensor, a sound sensor, a pressure regulator, and a processor; wherein
the pressure regulator is connected with the cuff and is configured to regulate a pressure applied to the measured position by the cuff;
the pressure sensor is arranged on the cuff and is configured to acquire the pressure applied to the measured position by the cuff during a period of time when the pressure is changed;
the sound sensor is positioned between the cuff and the measured position in a measuring process, and is configured to acquire blood flow sound at the measured position; and
the processor is communicated with the pressure sensor and the sound sensor, and is configured to determine a blood pressure value of the user according to acquisition results of the pressure sensor and the sound sensor.

2. The blood pressure measurement instrument according to claim 1, wherein the blood flow sound includes Korotkoff sound, and the blood pressure measurement instrument is configured to acquire pressure values respectively corresponding to an appearance moment and a disappearance moment of the Korotkoff sound during the period of time when the pressure is changed so as to determine the blood pressure value.

3. The blood pressure measurement instrument according to claim 2, wherein the processor includes a Korotkoff sound processing unit, the Korotkoff sound processing unit including a peak value acquiring module and a peak value comparing module;
the peak value acquiring module is configured to acquire a sound intensity peak value of the Korotkoff sound at the measured position in each period; and
the peak value comparing module is connected with the peak value acquiring module, and is configured to, according to acquisition results of the peak value acquiring module, compare a peak value in a current period with a peak value in a previous period.

4. The blood pressure measurement instrument according to claim 3, wherein the Korotkoff sound processing unit further includes a maximum peak value determining module,
the maximum peak value determining module is connected with the peak value comparing module and configured to, when the peak value comparing module concludes that the peak value in the current period is smaller than the peak value in the previous period for a first time, determine the peak value in the previous period as a maximum peak value of the Korotkoff sound, wherein the current period in which the maximum peak value of the Korotkoff sound is determined is referred to as a reference period.

5. The blood pressure measurement instrument according to claim 4, wherein the Korotkoff sound processing unit further includes a node determining module,
the node determining module is connected with the peak value comparing module and configured to, when the peak value comparing module concludes that the peak value in the current period is greater than or equal to 105% to 110% of the peak value in the previous period for the first time, determine a moment when the peak value in the current period appears as the appearance moment of the Korotkoff sound.

6. The blood pressure measurement instrument according to claim 5, wherein the node determining module is further connected with the maximum peak value determining module, and is configured to, when the peak value comparing module compares a peak value in each period after the reference period with the maximum peak value of the Korotkoff sound until the peak value comparing module obtains one period in which a peak value of this one period is smaller than or equal to 65% to 75% of the maximum peak value of the Korotkoff sound, determine a moment when the peak value in the one period appears as the disappearance moment of the Korotkoff sound.

7. The blood pressure measurement instrument according to claim 3, wherein the processor further includes a pressure matching unit;
the pressure matching unit is connected with the Korotkoff sound processing unit and the pressure sensor, and is configured to receive the appearance moment and the disappearance moment of the Korotkoff sound, which are output by the Korotkoff sound processing unit, acquire a pressure value acquired by the pressure sensor at the appearance moment of the Korotkoff sound as a systolic blood pressure and acquire a pressure value acquired by the pressure sensor at the disappearance moment of the Korotkoff sound as a diastolic blood pressure.

8. The blood pressure measurement instrument according to claim 1, wherein the pressure regulator includes an air pump, an air delivery pipe connected with the air pump, and an airbag connected to the other end of the air delivery pipe,
the airbag is arranged inside the cuff, and the air pump is configured to inflate and deflate the airbag by the air delivery pipe.

9. The blood pressure measurement instrument according to claim 8, wherein the processor further includes a pressure controlling unit;
the pressure regulator further includes a regulation valve mounted on the air pump, and the regulation valve is connected with the pressure controlling unit and is configured to regulate a pressure in the air pump under control of the pressure controlling unit,

10. (canceled)

11. (canceled)

12. (canceled)

13. A Korotkoff sound identifying method for acquiring blood flow sound including Korotkoff sound at a measured position, comprising:
acquiring a sound intensity peak value of the Korotkoff sound in each period;
comparing a peak value in a current period with a peak value in a previous period;
regulating a pressure at the measured position; and
determining an appearance moment, a maximum peak value and a disappearance moment of the Korotkoff sound.

14. The method according to claim 13, wherein
when the peak value in the current period is greater than or equal to 105% to 110% of the peak value in the previous period for a first time, a moment when the peak value in the current period appears is determined as the appearance moment of the Korotkoff sound.

15. The method according to claim 13, wherein when the peak value in the current period is smaller than the peak value in the previous period for a first time, the peak value in the previous period is determined as the maximum peak value of the Korotkoff sound, wherein a period in which the maximum peak value of the Korotkoff sound is determined is referred to as a reference period.

16. The method according to claim 13, wherein
in a case of comparing a peak value in each period after the reference period with the maximum peak value of the Korotkoff sound, when one period in which a peak value of this one period is smaller than or equal to 65% to 75% of the maximum peak value of the Korotkoff sound is compared out, a moment when the peak value in the one period appears is determined as the disappearance moment of the Korotkoff sound.

17. The Korotkoff sound identifying method according to claim 15, wherein a process that when the peak value in the current period is smaller than the peak value in the previous period for the first time, the peak value in the previous period is determined as the maximum peak value of the Korotkoff sound, the period in which the maximum peak value of the Korotkoff sound being determined is referred to as the reference period, includes:
when the peak value in the current period is smaller than the peak value in the previous period for the first time, referring to the previous period as a first period, and referring to the current period as a second period;
after the second period, at least carrying out an operation of comparing a peak value in a new current period with a peak value in a new previous period; and
when a comparison result of the above operation each shows that the peak value in the new current period is smaller than the peak value in the new previous period, determining the first period as the reference period.

18. The Korotkoff sound identifying method according to claim 13, wherein before the appearance moment of the Korotkoff sound, regulating the pressure at the measured position includes: pressurizing the measured position at a speed of 2 to 4 mmHg/s; and when the pressure at the measured position reaches 210 mmHg to 260 mmHg, releasing the pressure at the measured position at a speed of 2 to 4 mmHg/s;
or, in two to three seconds after the disappearance moment of the Korotkoff sound, regulating the pressure at the measured position further includes: releasing the pressure at the measured position at a speed of 8 to 12 mmHg/s.

19. An apparatus for carrying out Korotkoff sound identification by adopting the Korotkoff sound identifying method according to claim 13, comprising a Korotkoff sound processing unit, the Korotkoff sound processing unit including a peak value acquiring module, a peak value comparing module, a maximum peak value determining module and a node determining module;
the peak value acquiring module is configured to acquire a sound intensity peak value of Korotkoff sound at a measured position in each period;
the peak value comparing module is connected with the peak value acquiring module, and is configured to, according to acquisition results of the peak value acquiring module, compare a peak value in a current period with a peak value in a previous period;
the maximum peak value determining module is connected with the peak value comparing module, and is configured to determine a maximum peak value of the Korotkoff sound;
the node determining module is connected with the peak value comparing module, and is configured to determine an appearance moment of the Korotkoff sound; and
the node determining module is further connected with the maximum peak value determining module, and is configured to determine a disappearance moment of the Korotkoff sound.

20. The apparatus according to claim 19, wherein the maximum peak value determining module is configured to, when the peak value comparing module concludes that the peak value in the current period is smaller than the peak value in the previous period for a first time, determine the peak value in the previous period as a maximum peak value of the Korotkoff sound, wherein the period in which the maximum peak value of the Korotkoff sound is determined is referred to as a reference period.

21. The apparatus according to claim 19, wherein the node determining module is configured to, when the peak value comparing module concludes that the peak value in the current period is greater than or equal to 105% to 110% of the peak value in the previous period for the first time, determine a moment when the peak value in the current period appears as the appearance moment of the Korotkoff sound.

22. The apparatus according to claim 19, wherein the node determining module is configured to, when the peak value comparing module compares a peak value in each period after the reference period with the maximum peak value of the Korotkoff sound until the peak value comparing module obtains one period in which a peak value of this one period is smaller than or equal to 65% to 75% of the maximum peak value of the Korotkoff sound, determine a moment when the peak value in the one period appears as the disappearance moment of the Korotkoff sound.

23. The apparatus according to claim 19, further comprising a pressure controlling unit,
the pressure controlling unit is configured to: before the appearance moment of the Korotkoff sound, pressurize the measured position at a speed of 2 to 4 mmHg/s; when a pressure at the measured position reaches 210 mmHg to 260 mmHg, release the pressure at the measured position at a speed of 2 to 4 mmHg/s; and in two to three seconds after the disappearance moment of the Korotkoff sound, release the pressure at the measured position at a speed of 8 to 12 mmHg/s.
